(19) United States
(12) Patent Application Publication
(10) Pub. No.: US 2008/0109021 A1
(43) Pub. Date: May 8, 2008

(54) SURGICAL TOOL WITH INTEGRAL BLADE AND SELF-CENTERING TIP

(76) Inventor: Robert J. Medoff, Kailau, HI (US)

Correspondence Address: PATZIK, FRANK & SAMOTNY LTD., 150 SOUTH WACKER DRIVE, SUITE 1500, CHICAGO, IL, 60606, US

(21) Appl. No.: 11/557,414

(22) Filed: Nov. 7, 2006

(51) Int. Cl. A61B 17/3209

(52) U.S. Cl. 606/167

(57) ABSTRACT

A surgical tool for division of a tendon sheath having a convex outer surface, such as the A1 pulley of the finger, is disclosed. A cutting tip is configured to self-center itself and, in turn, a cutting blade carried by the cutting tip, as the cutting tip is advanced along the arcuate outer surface of the tendon sheath. The cutting tip is further configured to displace neural tissue and vascular tissue from a region proximate the cutting blade as the cutting tip is advanced along the arcuate outer surface of the tendon sheath. A retractable blade guard is disclosed. The cutting blade may be fixed, relative to the cutting tip. Alternatively, the cutting blade may be moveable from a retracted position and a deployed position, and vice-versa.

### BACKGROUND OF THE INVENTION

1. Field of the Invention

The invention relates to a surgical instrument to allow division of a tendon sheath in a safe and simplified manner.

Swelling around tendons may lead to pathologic conditions in areas where the tendons traverse a contained space. For instance, the condition of stenosing tenosynovitis in the hand can produce the common condition known as a trigger finger in which an area of irritation or a nodule in the tendon becomes trapped as it passes through the fibrous tunnel known as the A1 pulley in the hand. This may lead to binding of the tendon and interference with movement of the fingers, resulting in the locking and unlocking of the finger as it is flexed that gives the condition its name. Stenosing tenosynovitis of the thumb abductor tendon can cause pain at the wrist and is known as DeQuervain's syndrome; pathologically, it is caused by a similar condition. When swelling occurs within the confines of the carpal tunnel, the increased pressure may lead to dysfunction of the median nerve as it is squeezed against the undersurface of the transverse carpal ligament. When these conditions do not respond to non-surgical methods of treatment, operative intervention may be required. In the case of carpal tunnel syndrome, release of the transverse carpal ligament is performed. In the case of a trigger finger, surgical release of the A1 pulley of the flexor tendon is done.

Although the pathology of carpal tunnel syndrome and trigger finger are similar, the anatomic features of these two conditions are quite different and can affect the technique of the surgical release. The transverse carpal ligament that runs across the base of the wrist is flat and applying pressure from a knife on the top or edge of the ligament does not cause the blade to slip to either side. In contrast, the flexor sheath of the A1 pulley system is cylindrical, and applying a knife to the top of the sheath results in a tendency for the blade to slip off to one side. At the carpal tunnel, the contents of the enclosed space contain the median nerve which is located along the undersurface of the ligament. This important structure is at risk when dividing the ligament, and care must be taken to separate the nerve from the undersurface of the ligament and protect it from injury by a knife during division of the ligament. Dividing the ligament by cutting it downward from the superficial surface with a knife blade, particularly if done without direct visualization, can risk cutting this nerve. There are no major nerves or vessels immediately superficial, to the transverse carpal ligament. In contrast, in the case of trigger finger, there are no important nerves or vessels that require protection within the contents of the sheath, instead, there are important digital nerves and arteries adjacent to the superficial surface of the sheath that run along either side. Care must be taken to protect these superficial neurovascular structures so that a knife blade does not inadvertently slip to one side and cut them.

Because of the close proximity of the digital nerves on the superficial surface of the tendon sheath, surgical release for trigger linger is most commonly done with an open technique. This involves creating an incision large enough, for placement of retractors in order to protect the neurovascular structures along the sides of the sheath and directly visualize the A1 pulley; the sheath can be then safely divided under direct vision longitudinally with a knife. Since this open technique requires special retractors, adequate lighting, sterile instrumentation, and effective anesthesia, it is typically done in a standard operating suite or outpatient surgical center. This adds considerable expense and time commitment for the patient. In addition, since the surgical procedure in these locations is often classified by insurance carriers as containing a facility charge, the surgical fee to the surgeon is substantially reduced from an identical procedure that is done in an office setting. In addition, the surgeon must spend additional time and office resources to generate, process and deliver the extra required paperwork that is involved in performing procedures within a facility setting.

2. Description of Related Art

U.S. Pat. Nos. 5,269,796, 5,282,81.6, 5,273,024, 5,325,883, 5,387,222, 5,507,800, 5,45.5,8611, 5,651,790, 5,569,283, 5,649,946, 5,827,311, 5,908,433, 6,019,774, 6,179,852, 6,019,774 and U.S. patent application Ser. No. 004/0054378 each describe instruments and methods for dividing the transverse carpal ligament when doing a carpal tunnel release. Such prior art techniques and instruments used to release a retinacular ligament or sheath are not necessarily suited for division of a retinacular sheath such as the A1 pulley that is involved with trigger finger.

U.S. Pat. Nos. 4,962,770, 4,963,1.47, 5,089,000, 5,306,284, 5,968,061, and 7,041,115 likewise describe, in general, arthroscopic techniques to divide the transverse carpal ligament for carpal tunnel syndrome. Arthroscopy has limited applicability for division of the A1 flexor sheath for trigger finger, due as the very small size of the tendon sheath, and the risk of clipping a digital nerve from inaccurate placement of the arthroscope. In addition, arthroscopic techniques often require a high degree of surgical expertise and often require relatively bulky, expensive equipment and, accordingly, are not well suited for use in an office environment.

U.S. Pat. Nos. 5,029,573, 5,346,503, 5,356,4.19, 5,480,408, and 5,353,812; and U.S. patent application number 2004/0098005 disclose, in general, arthroscopic instruments and techniques using an edge cutting knife.

U.S. patent application number 2006/0190021 discloses a method for dividing the A1 pulley for trigger finger in which a hollow bored cannula is passed through the skin and subcutaneous tissue. The implant has a wire tip than can be deployed to extend from the end of the bore and is used to puncture into the tendon sheath. The instrument has a recessed edge cutting blade which is then advanced, relying the wire tip to remain within the flexor sheath and guide the blade along the edge of the sheath to divide it. Because of the proximity of the digital nerves on the superficial surface of the flexor sheath, it appears to be possible to wrap a nerve across the advancing tip of the instrument, potentially resulting in a nerve transection with permanent loss of feeling in the finger. Moreover, unless this technique is used with enough dissection to allow visualization, the surgeon may not be sure that he or she has entered the sheath all the way at one end in order to ensure complete division of the tendon sheath. Finally, since the tip disclosed is cylindrical in shape, there may be a tendency for the instrument to tend to be displaced to one side of the convex surface of the flexor sheath, risking potential iatrogenic injury to a digital nerve.

U.S. Pat. No. 5,957,944 describes a hooked pin that is placed directly into a flexor tendon through the flexor sheath. The pin has a knife blade on one edge so that as the patient moves the tendon through the sheath with flexion/extension of the finger, the pin slides proximally and distally, theoretically resulting in division of the ligament. However, movement of the flexor tendons may not reliably result in complete division of the sheath from its proximal edge to its distal edge. In addition, movement of the tendons may cause the pin to simply rotate to accommodate the movement, and may not result in division of the flexor sheath. Finally, since the sheath is cylindrical in shape being convex dorsally, there may be a tendency for the instrument to be laterally displaced to one side during insertion, which may place the cutting pin outside of the sheath and off to one side.

U.S. Pat. No. 6,685,717 discloses instrument with a flat surface from which a blade extends at a right angle. This instrument is intended to be placed under the transverse carpal ligament and advanced down the ligament, cutting the ligament from the bottom starting at one edge while displacing and protecting the median nerve from the undersurface of the ligament while the blade is advanced. This instrument is not as applicable in the case of a trigger finger, since the digital nerves run above the flexor sheath and could be at risk for division by the blade. Moreover, the edge cutting nature of the blade may put the nerves and arteries at risk of being kinked, tied up and divided when advancing this edge cutting knife down the tendon sheath. Furthermore, there appears to be insufficient room within the Al tendon sheath to allow placement of this instrument. Also, unlike the transverse carpal ligament which is flat, this risk is heightened in division of the flexor sheath in the finger, since the convex nature of the surface of the sheath would to displace the knife blade off to one side or the other of the sheath.

U.S. Pat. No. 5,613,976 describes a method in which the transverse carpal ligament is clamped along its deep and superficial surface by two jaws of an instrument. A knife is then passed along the deep side and pressure from above pushes the ligament against the blade. This method requires exposure of the ligament for proper placement of the two jaws of the guide, as well as dissection on both the superficial and deep surfaces of the ligament in order to place the jaws into the correct position. It is, accordingly, not particularly well suited for application to the A1 pulley of the flexor tendon sheath of the finger, inasmuch as there is insufficient room for placement of this type of jaw, and the required exposure is extensive.

Accordingly, it is an object of the present invention to provide an instrument for division of a cylindrical shaped tendon sheath such as the A1 pulley, it is another object of the present invention to provide a surgical instrument that permits the A1 pulley to be divided in an office setting, without requiring extensive retractors, lighting and assistance. It is yet another object of the present invention to provide a surgical instrument which displaces and protects the superficial digital nerves and arteries that run on either side of the flexor sheath while allowing division of the flexor sheath. It is still another object of the present invention is to provide a surgical instrument which allows division of a tendon sheath with a cutting blade that can be pressed from the superficial surface of the sheath downwards, but with a stop or block that prevents the knife from plunging excessively deep into the contents of the sheath. It is also an object of the present invention to provide a surgical tool having a surface contour to the instrument that tends to center the blade on the top of the flexor sheath and avoids lateral slippage off to one side or the other.

### BRIEF SUMMARY OF THE INVENTION

The present invention is a surgical tool for making controlled incisions in subcutaneous structures. The surgical tool comprises a handle region, and a cutting tip operably attached to the handle region. The cutting tip has a proximal end proximate the handle region, a distal end, a longitudinal axis extending from the proximal end to the distal end, and an upper and a lower surface. The upper surface is either substantially convex or substantially torispherical (i.e., the outer surface of a torispherical dome). The cutting tip further includes a longitudinal cavity defined by at least a portion of its lower surface.

A cutting blade is provided. The cutting blade has a deployed position wherein the cutting blade extends along at least a portion of the longitudinal axis of the cutting tip and extends downwardly from the bottom surface of the cutting tip into at least a portion of the longitudinal cavity.

In a preferred embodiment, the cutting blade is moveable back and forth between the deployed position and a retracted position. Substantially the entire cutting blade is removed from the longitudinal cavity of the cutting tip when the cutting tip is in its retracted position.

In one preferred embodiment, movement of the cutting blade between the retracted and deployed positions is in a direction substantially perpendicular to the longitudinal axis of the cutting tip. In another preferred embodiment, movement of the cutting blade between the retracted and deployed positions is in a direction substantially along the longitudinal axis of the cutting tip. Moreover, in a preferred embodiment, substantially the entire cutting blade is carried the cutting tip, the handle region, or a combination of the cutting tip and the handle region, when the cutting tip is in the retracted position.

In a preferred embodiment, the surgical tool further comprises a pushbutton operably attached to the cutting blade. Depression of the pushbutton causes the cutting blade to move from its retracted position to its deployed position. At least a portion of the pushbutton may be integrally formed with the cutting blade.

In another preferred embodiment, a button is operably attached to the cutting blade and slidably carried within the cutting tip, the handle region, or a combination of the cutting tip and the handle region. Sliding movement of the button from a first position to a second position causes the cutting blade to move from the retracted position to the deployed position.

A blade guard is optionally provided. The blade guard is carried within the cutting tip, the handle region, or a combination of the cutting tip and handle region. The blade guard is moveable from a guarded position to an unguarded position. In the guarded position, the blade guard covers at least a portion of a cutting edge of the cutting blade. In the unguarded position, at least a portion of the cutting edge of the cutting blade is exposed. A button may be operably attachable to the blade guard and slidably carried within the cutting tip, the handle region, or a combination of the cutting tip and handle region. Sliding movement of the button from a first position to a second position causes the blade guard to move from its guarded position to its unguarded position.

In a preferred embodiment, the upper surface of the cutting tip is substantially convex, and the cutting tip Is substantially ere scent-shaped in cross-section. In another preferred embodiment, the cutting tip comprises an elongated spherical cap region, a first elongated cylindrical region, and a second elongated cylindrical region. In this embodiment, the upper surface of the overall cutting tip is substantially torispherical and is comprised of at least a portion of the spherical cap region, the first elongated cylindrical region, and the second elongated cylindrical region. The lower surface of the tip is comprised of at least a portion of the spherical cap region, the first elongated cylindrical region, and the second elongated cylindrical region. The cutting blade extends along at least a portion of a longitudinal axis of the spherical cap and extends downwardly from a bottom surface of the spherical cap region of the cutting tip. In one preferred embodiment, the first and second cylindrical regions of the cutting tip are joined in a loop proximate the distal end of the cutting tip. In another preferred embodiment, the first and second cylindrical regions of the cutting tip meet to formed a tapered end proximate the distal end of the cutting tip.

Moreover, in a preferred embodiment, the surgical tool further includes a dissection tip operably coupled to the handle region. The dissection tip is preferably substantially similar in configuration to the cutting tip.

The present invention also comprises a surgical tool for making subcutaneous incisions through at least a portion of a tendon sheath having a substantially arcuate outer surface. The surgical tool comprises a handle region and a cutting tip operably attached to the handle region. The cutting tip has a proximal end proximate the handle region, a distal end, a longitudinal axis extending from the proximal end to the distal end, an upper and a lower surface, and a longitudinal cavity defined by at least a portion of the lower surface. The surgical tool further includes a cutting blade having a deployed position wherein the cutting blade extends along at least a portion of the longitudinal axis of the cutting tip and extends downwardly from the bottom surface of the cutting tip into at feast a portion of the longitudinal cavity. In this preferred embodiment, the lower surface of the cutting tip is configured to self-center the cutting tip as the cutting tip is advanced along the arcuate outer surface of the tendon sheath.

The present invention also comprises a surgical tool for making subcutaneous incisions through at least a portion of a tendon sheath proximate at least one of neural tissue and vascular tissue. The surgical tool includes a handle region and a cutting tip operably attached to the handle region. The cutting tip has a proximal end proximate the handle region, a distal end, a longitudinal axis extending from the proximal end to the distal end, an upper and a lower surface, and a longitudinal cavity defined by at least a portion of the lower surface. The surgical tool also includes a cutting blade having a deployed position wherein the cutting blade extends along at least a portion of the longitudinal axis of the cutting tip and extends downwardly from the bottom surface of the cutting tip into at least a portion of the longitudinal cavity. The upper surface of the cutting tip is configured to displace at least one of the neural tissue and vascular tissue from a region proximate the cutting blade as the cutting tip is advanced along the arcuate outer surface of the tendon sheath.

### BRIEF DESCRIPTION OF THE SEVERAL VIEWS OF THE DRAWINGS

FIG. 1 of the drawings is simplified cross-sectional view of a portion of the human hand, proximate the A1 pulley flexor tendon sheath;

FIG, 2 of the drawings is a simplified cross-sectional view of the portion of the human hand of FIG, 1, showing, in particular, the release of A1 pulley by an embodiment of the present surgical tool;

FIG. 3A is a side elevational view of a first embodiment of a surgical tool embodying the present invention:

FIG. 3B is a side sectional view of the surgical tool of FIG. 3A; FIG. 3C is a sectional view of a portion of the surgical tool of FIG, 3A, taken generally along lines 3C-3C of FIG. 3A;

FIG. 3D is a sectional view of a portion of the surgical tool of FIG. 3A, taken generally along fines 3D-3D of FIG. 3A;

FIG. 4A is a side elevational view of a second embodiment of a surgical tool embodying the present invention;

FIG. 4B is a side sectional view of the surgical tool of FIG, 4A;

FIG. 4C is a sectional view of a portion of the surgical tool of FIG. 4A, taken generally along lines 4C-4C of FIG. 4A and showing, in particular, the blade guard in its guarded position;

FIG. 4D is a sectional view of a portion of the surgical tool of FIG. 4A, taken generally along lines 4C-4C of FIG. 4A and showing, in particular, the cutting blade when the blade guard is moved to its unguarded position;

FIG. 5A is a side elevational view of a third embodiment of a surgical tool embodying the present invention;

FIG. 5B is a side sectional view of the surgical tool of FIG. 5A showing, in particular, the cutting blade in its retracted position;

FIG, 5C is a fragmentary side sectional view of the surgical tool of FIG. 5A showing, in particular, the cutting blade in its deployed position;

FIG. 5D is a sectional view of a portion of the surgical tool of FIG. 5A, taken generally along lines 5D-5D of FIG. 5A and showing, in particular, the cutting blade in its retracted position;

FIG. 5E is a sectional view of a portion of the surgical tool, of FIG. 5A taken generally along lines 5E-5E of FIG. 5C and showing, in particular, the cutting blade in its deployed position;

FIG. 6A is a side elevational view of a fourth embodiment of a surgical tool embodying the present invention;

FIG. 6B is a side sectional view of the surgical tool of FIG. 6A showing, in particular, the cutting blade in its retracted position;

FIG. 6C is a fragmentary side sectional view of the surgical tool of FIG. 6A showing, in particular, the cutting blade in its deployed position;

FIG. 7A is a side elevational view of a fifth embodiment of a surgical tool embodying the present invention showing, in particular, the cutting blade in its retracted position;

FIG. 7B is a side sectional view of the surgical tool of FIG. 7A showing, in particular, the cutting blade in its deployed position;

FIG. 7C is a sectional view of the surgical tool of FIG. 7A, taken generally along lines 7C-7C of FIG, 7A and showing, in particular, the cutting blade in its retracted position;

FIG. 7D is a sectional view of the surgical tool of FIG. 7A, taken generally along lines 7D-7D of FIG. 7B and showing, in particular, the cutting blade in its deployed position;

FIG. 8A is a side elevational-view of a sixth embodiment of a surgical tool embodying the present invention and showing, in particular, the cutting blade in its retracted position;

FIG. 8B is a fragmentary top plan view of the surgical tool of FIG. 8A;

FIG. 8C is a fragmentary side sectional view of the surgical tool of FIG. 8A showing, in particular, the cutting blade in its deployed position;

FIG. 8D is a sectional view of the surgical tool of FIG. 8A, taken generally along lines 8D-8D of FIG. 8A and showing, in particular, the cutting blade in its retracted position;

FIG. 8E is a sectional view of the surgical tool of FIG. 8A, taken generally along lines 8E-8E of FIG. 8C and showing, in particular, the cutting blade in its deployed position;

FIG. 9A is a side elevational view of a seventh embodiment of a surgical tool embodying the present invention;

FIG. 9B is a fragmentary top plan view of the surgical tool of FIG. 9A and showing, in particular, the cutting blade in its guarded position;

FIG. 9C is a sectional view of the surgical tool of FIG. 9A, taken generally along lines 9C-9C of FIG, 9B and showing, in particular, the cutting blade in its guarded, position;

FIG. 9D is a fragmentary top plan view of the surgical tool of FIG. 9A and showing, in particular, the cutting blade in its deployed position;

FIG. 9E is a sectional view of the surgical tool of FIG. 9A, taken generally along lines 9E-9E of FIG. 90 and showing, in particular, the cutting blade in its deployed position;

FIG. 10A is a side perspective view of an eighth embodiment of a surgical tool embodying the present invention showing, in particular, the cutting blade in its guarded position;

FIG 10B is a sectional view of the surgical tool of FIG. 10A, taken generally along lines 10B-10B of FIG. 10A and showing, in particular, the cutting blade in its guarded position;

FIG. 10C is a side perspective view of the surgical tool, of FIG. 10A showing, in

particular, the cutting blade in its deployed position; and

FIG. 10D is a sectional view of the surgical tool of FIG. 10C, taken generally along lines 10D-10D of FIG. 10C and showing, in particular, the cutting blade in its deployed position.

### DETAILED DESCRIPTION OF THE INVENTION

While several different preferred embodiments are shown in the various FIGS., common reference numerals in the figures denote similar or analogous elements or structure amongst the various embodiments. Moreover, in the figures, the lighter shaded regions indicate structure and/or elements which are hidden from view behind other structure or elements.

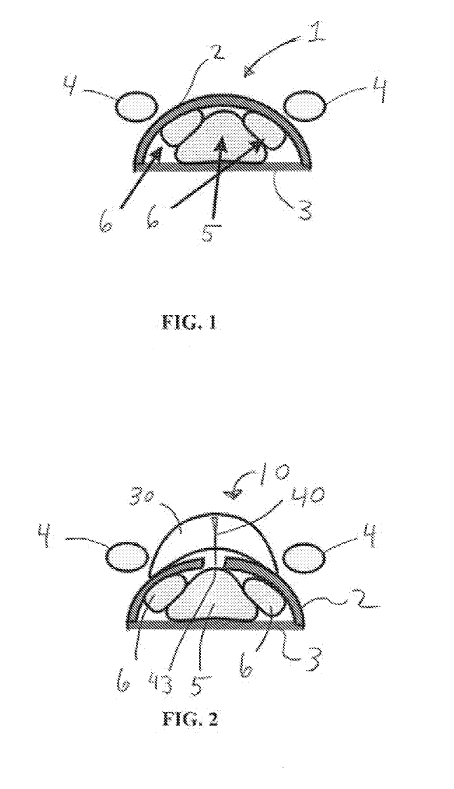

Referring to FIG. 1, a partial, transverse cross-section of the human hand, proximate the junction of metacarpal and proximal phalanx bones, is shown in simplified form. In particular, the A1 pulley tendon sheath 1 is shown, having a relatively planar floor 3 adjacent metacarpal and proximal phalanx bones (not shown), and an convex, arcuate outer surface 2 extending outwards towards the palm of the hand. The flexor digitorum profundus (FDP) tendon 5 and flexor digitalis superficialis (FDS) tendons 6 are encased within tendon sheath 1 from a region that partially overlies a metacarpal bone extending longitudinally to a region that partially overlies a proximal phalanx bone. Digital nerves 4 extend along the exterior of the A1 pulley, adjacent opposing sides of a vertical centerline of the arcuate outer surface 2 of the A1 pulley 1.

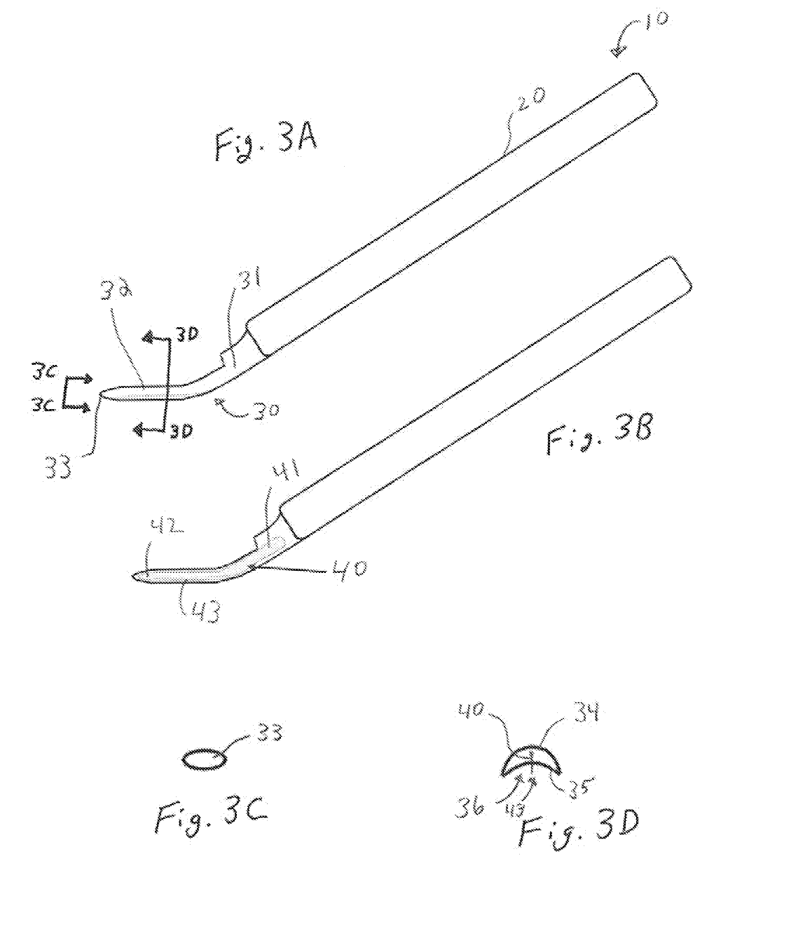

A surgical tool of the present invention, representative of any of the embodiments of FIGS. 3-7, is shown in operation In FIG. 2. Surgical tool 10 includes a cutting tip 30 which, in several preferred embodiments, has a generally crescent shaped cross section. As shown in FIG. 2, cutting tip 30 has a convex upper surface, a concave lower surface that is substantially elliptical, forming two opposing wing-like members on opposite sides of cutting blade 40. Cutting blade 40 extends vertically downwards from the lower surface of the cutting tip, centered vertically between the two wing-like members. In particular, the concave, substantially elliptical lower surface of the cutting tip forms a longitudinal channel, with the cutting blade extending downwards into the longitudinal channel.

As shown in FIG. 2, the concave, substantially elliptical lower surface of cutting tip 30 is sized and shaped to approximate the arcuate outer surface 2 of the A1 tendon sheath. This tends to self-center the cutting tip and, in turn, cutting blade 40 and cutting edge 43, about the longitudinal center of tendon sheath 1, as cutting tip 30 is contacted with the tendon sheath (i.e., advanced along the tendon sheath or pushed downward towards the sheath from above), and tends to inhibit the cutting tip from sliding laterally to either side as the cutting tip is advanced. Moreover, and as shown in FIG. 2, this further results in the severing of the convex tendon sheath proximate it's centered, top-most portion.

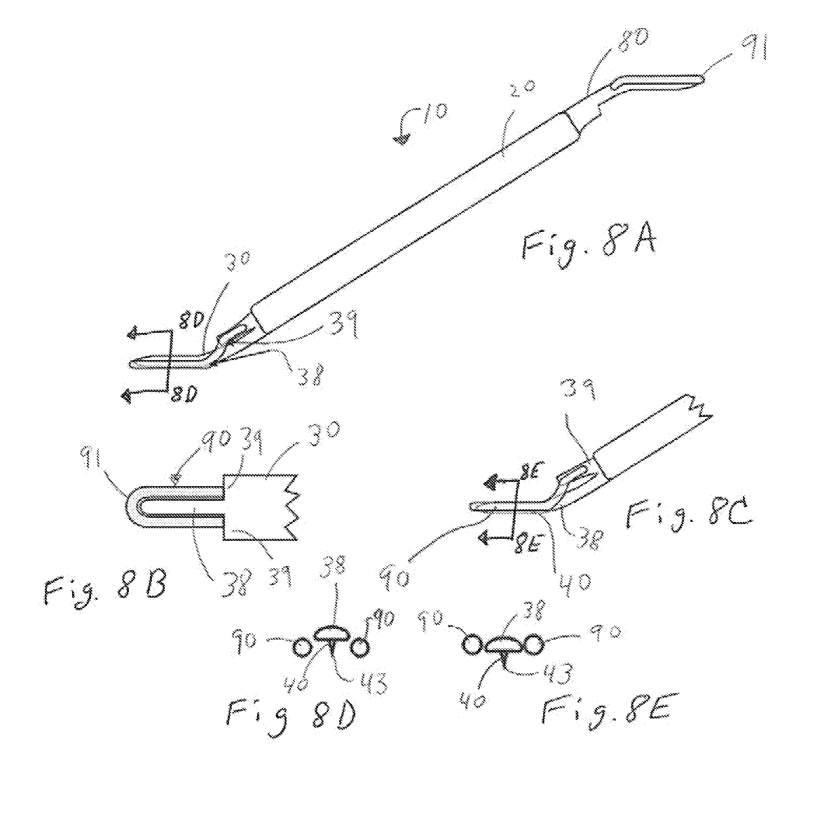

In FIG. 2, the lower surface of the cutting tip 30 is shown as being of integral construction with a symmetric elliptical surface. The shape and contour of this surface are optimally designed to approximate the surface topology of the tendon sheath, directing the instrument to self-center over the central axis of the tendon sheath. Although FIG. 2 shows a cutting tip of unitary, or integral construction with continuous contact between the undersurface of the cutting tip and the tendon sheath, alternate embodiments, also contemplated by the present invention, may be employed in which the side walls and central portion of the undersurface of the cutting tip are comprised of multiple, separate structures, such as, for example, the separate structures depicted in FIGS. 8-10. In addition, it is sufficient to limit contact between the undersurface of the cutting tip and the tendon sheath at two or more point locations, rather than require a zone of continuous contact. Finally, the specific dimensions of the shape of the undersurface of the cutting tip combined with the depth of the exposed blade, relative to both the longitudinal cavity of the cutting tip and the lower-most edges of the cutting tip (and, in turn, the depth of the longitudinal cavity) can be adjusted to provide a predetermined and controlled depth of incision of the blade, thereby avoiding inadvertent damage to the tendon structures within the sheath.

Furthermore, the two wing-like members tend to laterally displace the digital nerves 4 as the cutting tip is advanced lengthwise along the tendon sheath, inhibiting the digital nerves from becoming trapped inside the longitudinal channel beneath the lower surface of the cutting tip and, in turn, inhibiting the digital nerves from being inadvertently severed by the cutting blade. Although not shown in FIGS. 1 and 2, the digital arteries run in conjunction with the digital nerves and are also protected by the two wing-like members.

A first embodiment of surgical tool 10 is shown in FIGS. 3A-3D as comprising handle 20. Cutting tip 30 is operably attached to handle 20 at proximal end 31 of the cutting tip. Cutting tip 30 and handle 30 may comprise separate components affixed together or, alternatively, may be integrally formed from a single suitable material, such as surgical stainless steel, plastic, or other material. Moreover, cutting tip 30 may extend to form an interior portion of handle 20, with an exterior, gripping portion of handle 20 being a separate joined component.

Cutting tip 30 includes an elongated cutting region 32, tapered distal end 33, convex upper surface 34, and concave lower surface 35. Concave lower surface forms an interior, longitudinal cavity 36 along the length of elongated cutting region 32. An elongated cutting blade 40 operably attached to cutting tip 30, includes proximal end 41, distal end 42, and cutting edge 43, exposed along the length of longitudinal cavity 36. Cutting blade 40 may be integrally formed with cutting tip 30 or, alternatively, may be a separate, bonded component. As shown in FIG. 3D, cutting blade 40 extends vertically downwards from lower surface 35 of cutting tip 30, and extending into longitudinal cavity 36.

The tapered distal end of the cutting tip allows the instrument to dissect and develop a horizontal plane between the elastic soft tissues above the tendon sheath and the less elastic superficial surface of the tendon sheath itself. This horizontal plane of dissection occurs as the instrument is advanced longitudinally along the superficial surface of the tendon sheath and results in displacement of the neurovascular structures within the elastic soft tissues to a position along the superficial surface of the tapered end. As the instrument is advanced, the concave undersurface of the middle region of the cutting tip centers along the longitudinal axis of the sheath, maintaining the central axis of dissection. The convex surface of the middle region of the cutting tip results in additional lateral displacement of the neurovascular structures, further protecting them from contact with the knife blade.

In a preferred embodiment, elongated cutting region 32 is approximately 25.0 millimeters in length, and may, in other embodiments, vary from approximately 10 mm to approximately 40 mm in length. The overall height of cutting tip 30, from the junction of its lower and upper surfaces to the apex of the convex upper surface, is approximately 4.0 millimeters. The central thickness of the cutting tip 30, from the apex of its concave lower surface to the apex of its convex upper surface, is approximately 1.0-2.0 millimeters. Cutting blade 40, and in particular cutting edge 43, preferably extends vertically downwards approximately 1.3 millimeters into longitudinal cavity 36. Cutting edge 43 accordingly ends vertically approximately 0.7 millimeters above a horizontal plane intersecting the two parallel, longitudinal lines formed by the junctures of the upper and lower surfaces of cutting tip 30. In this manner, the cutting tip serves as a stop, or block, which prevents the cutting edge from plunging excessively deep into the contents of the tendon sheath and potentially cutting a portion of the FDP and IDS tendons within. In addition, in this embodiment, since the blade is recessed within the concave undersurface and does not extend beyond the sides of the cutting tip, the neurovascular structures along the side are protected from sliding under the edge of the blade.

The two longitudinal lines formed at the junctures of the upper and lower surfaces of cutting tip 30 are approximately 4.0 millimeters apart and, accordingly, cutting tip 30 has an overall width of approximately 4.0 millimeters at its widest part. Handle 20 is preferably approximately 140.0 millimeters in length and approximately 13.0 millimeters in cross-section. Handle 20 may be round, rectangular, or any other convenient shape in cross-section. To facilitate gripping, all or a portion of the surface of handle 20 may be knurled or ribbed. Of course, these measurements may be varied, without departing from the scope of the present invention, so as to adapt the present surgical tool for particular desired applications.

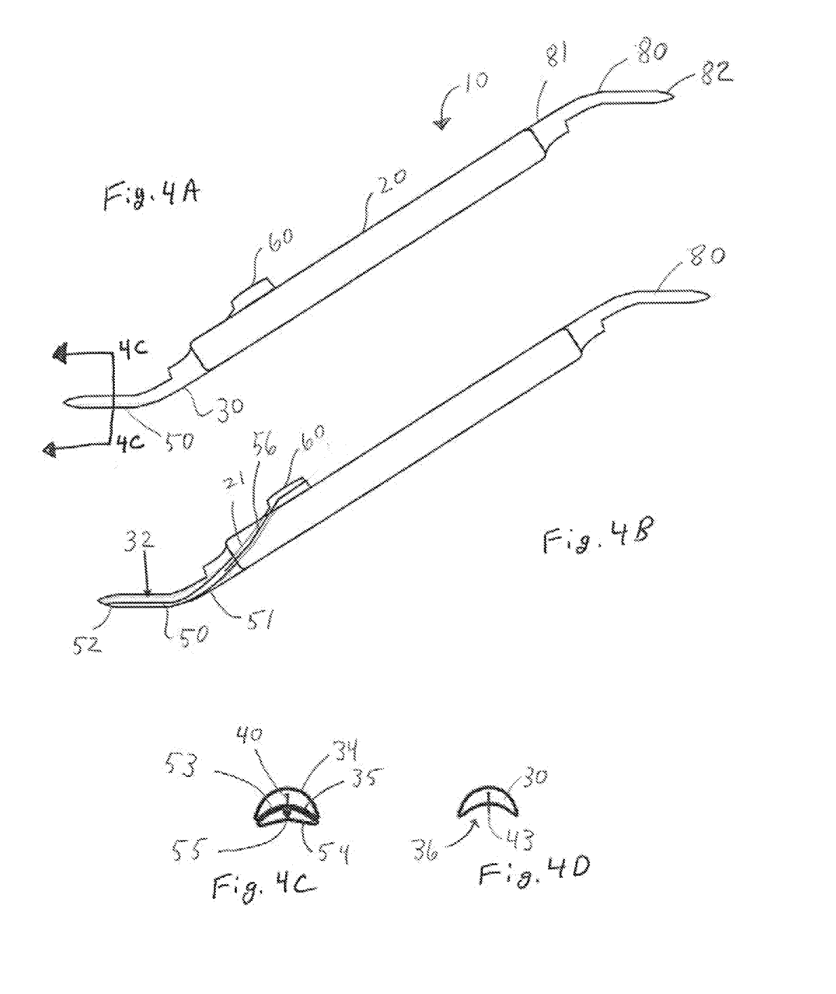

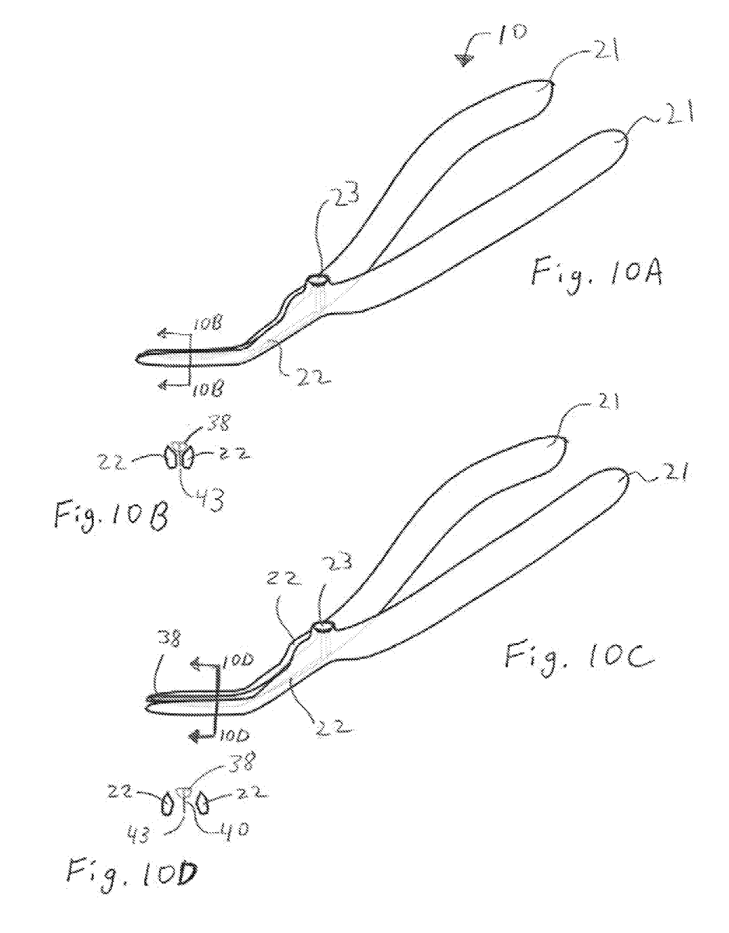

A second preferred embodiment of surgical tool 10 is shown in FIGS. 4A-4D. In this preferred embodiment, a dissection tip 80, including proximal end 81 and distal end 82, is operably attached to an opposing end of handle 20. Dissection tip 80 is preferably substantially similar is size and shape to cutting tip 30, with the exception that a cutting blade is absent from dissection tip 80. This permits the surgeon to use dissection tip 80 to develop a plane of dissection between the outer, convex surface of the tendon sheath and the soft tissue structures proximate the outer surface of the tendon sheath. Although dissection tip 80 is illustrated in connection with the embodiments of FIGS. 4, 7, 8 and 9, a second dissection tip could be used with any embodiment of the present invention. Moreover, for the embodiment of FIG. 10, multiple dissection tips may be employed in one, or both of the opposing handle arms.

Moreover, in this preferred embodiment, a blade guard 50 is provided. Blade guard 50 includes proximal end 51, distal end 52, upper surface 53, lower surface 54, longitudinal slot 55, and extender region 56. Sliding button 60 is operably attached to extender region 56 of blade guard 50, and is movable along a longitudinal slot of handle from a guarded position, proximate cutting tip 30 (as shown in FIGS. 4A-4B) to a distal region, farther from cutting tip 30. As shown in FIGS. 4A-4C, when sliding button 60 is in its guarded position, blade guard 50 is likewise in a guarded, position, wherein longitudinal slot 55 is adjacent cutting edge 43 of cutting blade 40, and blade guard 50 covers both blade 40 and longitudinal cavity 36 of cutting tip 30.

In operation, sliding button 60 and blade guard 50 are placed in their guarded positions as cutting tip 30 is inserted through an incision in the palm of the hand, and then longitudinally advanced through the incision to a region proximate the tendon sheath to be released. Then, as shown in FIG. 4D, sliding button 60 is then advanced from its guarded to its unguarded position. This causes blade guard 50 to be retracted into an interior channel of blade guard 30 and handle 20, sliding blade guard 50 from its guarded to its unguarded position. This, in turn, exposes cutting edge 43 of cutting blade 40 within longitudinal cavity 36 of cutting tip 30, permitting cutting blade 40 to release the tendon sheath by simple downward pressure of the cutting tip or advancement along the length of the tendon sheath.

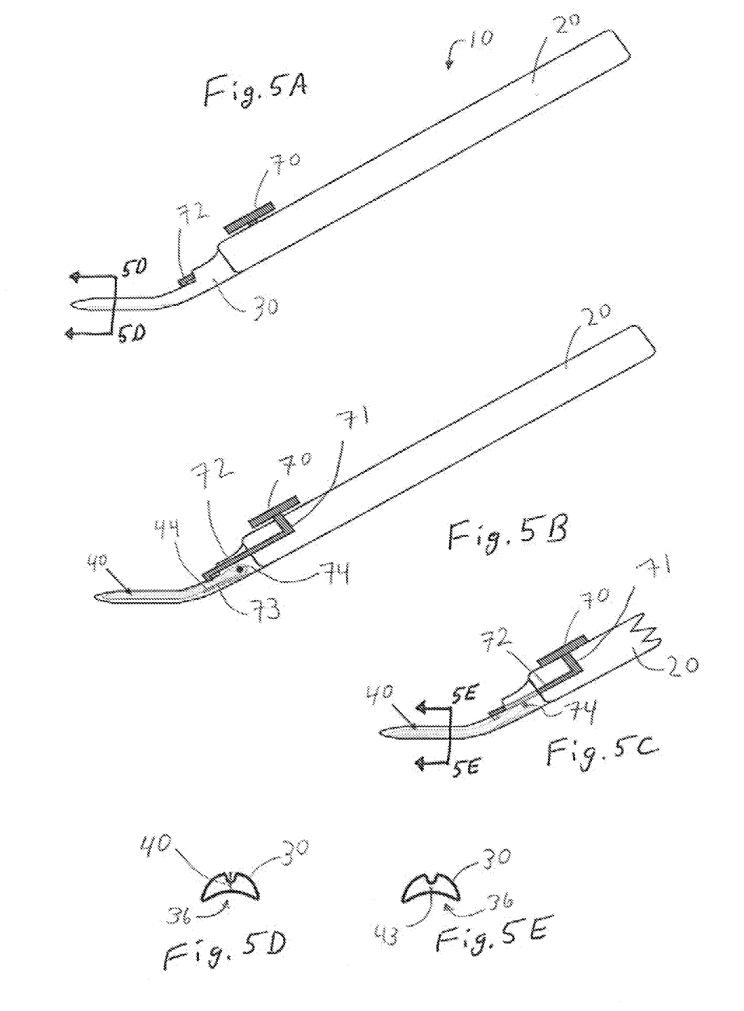

A third preferred embodiment of surgical tool 10 is shown in FIGS. 5A-5E. In this embodiment, cutting blade 40 is moveable between a retracted position, as shown in FIGS. 5A, 5B and 5D; and a deployed position, as shown in FIGS. 5C and 5E. In its retracted position, pushbutton 70 is elevated above the outer surface of handle 20. Leaf spring 73, carried within cutting tip 30 proximate its handle 20, biases an extension region 44 of cutting blade 40, causing the extender region and, in turn, the cutting region of cutting blade 40, to pivot about pivot pin 74 upwardly away from longitudinal cavity 36 of cutting tip 30. As best seen in FIG. 5D, in its retracted position, cutting edge 43 of cutting blade 40 is carried within a longitudinally extending, vertical slot of cutting blade 30. Moreover, a top, non-cutting portion of cutting blade 40 extends through a top opening of the vertical slot. As best seen in FIG. 5C, pressing downwards on pushbutton 70 towards handle 20 causes stem 71 to likewise move inwards along an interior cavity within handle 20. This, in turn, causes arm 72 to move cutting blade 40 downwards, about pivot pin 74, overcoming the opposing tension of leaf spring 73. As best seen in FIG. 5E, this causes cutting blade 40 to move from its retracted to its deployed position, wherein a portion of cutting blade 40, including cutting edge 43, extends into longitudinal cavity 36. Alternatively, if cutting blade 40 is constructed of a sufficiently spring-biased material, leaf spring 73 may be omitted, and pivot pin 74 may be replaced with a fixed attachment point of extension region 44 to an interior region of cutting tip 30 or handle 20.

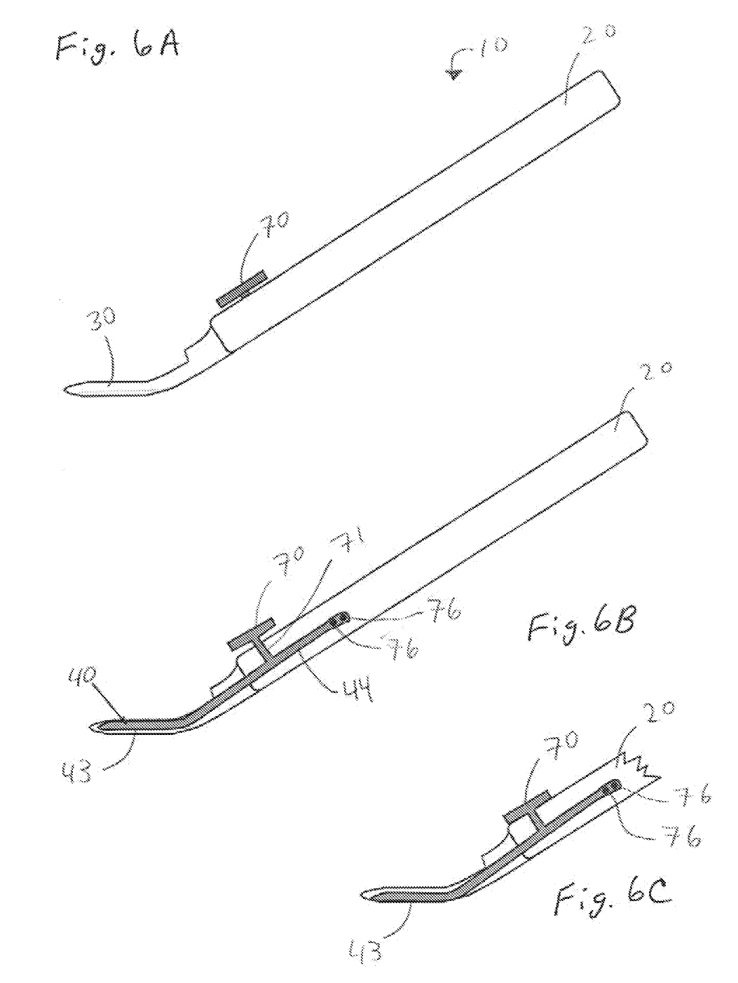

A fourth preferred embodiment of surgical tool 10 is shown in FIGS. 6A-6C. This embodiment is similar to the embodiment of FIGS. 5A-5E, except that the pushbutton 70 and stem 71 are integrally formed with cutting blade 40. In this embodiment, extension region 44 extends through a channel within both cutting tip 30 and handle 20, to attachment pins 76 carried within handle 20,

Cutting blade 20 is made of a sufficiently spring biased material that, as shown in FIG. 6C, when downward pressure is applied to pushbutton 70, cutting blade 40, and in particular, cutting edge 30, extends downwards from its retracted position, substantially within cutting tip 30, into its deployed position within longitudinal cavity 36 of cutting tip 30, as cutting blade 40 flexes proximate extension region 44. Release of pushbutton 70 allows cutting blade 40 to spring back into its retracted position. Alternatively, a pivot and leaf spring construction, such as in the embodiment of FIGS. 5A-5E, may alternatively be employed with the integral pushbutton and cutting blade of FIGS. 6A-6C.

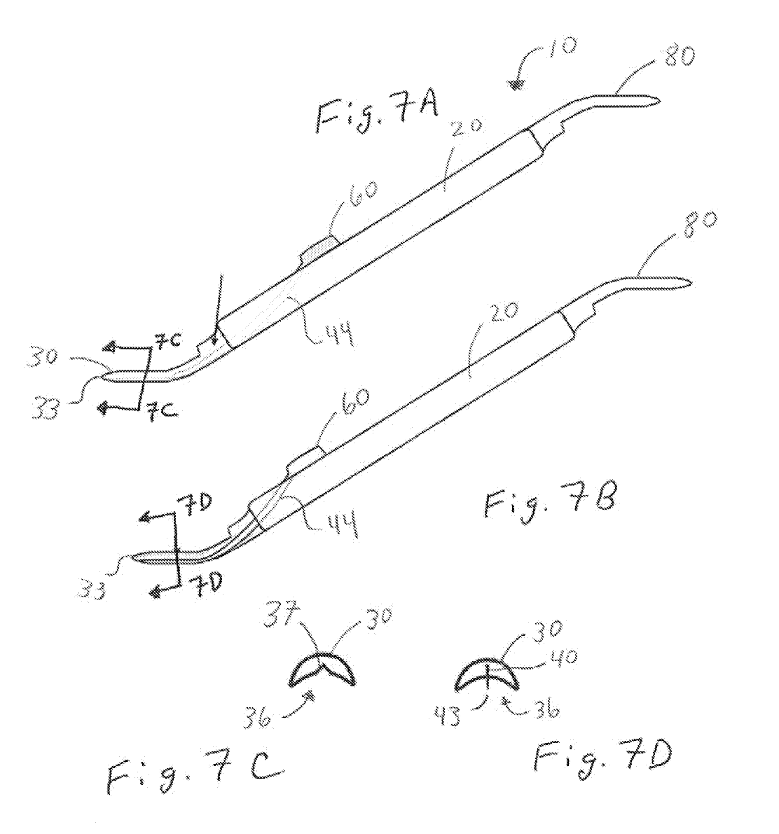

A fifth preferred embodiment of surgical tool 10 is shown in FIGS. 7A-7D in this embodiment, and in contrast to the embodiment of FIGS. 4A-4D, sliding of movement of button 60 causes blade 40 to move between retracted and deployed positions, rather than causing a blade guard to move between guarded and unguarded positions. In this embodiment, sliding button 60 is operably attached to extension region 44 of cutting blade 40. As shown in FIGS. 7A and 7C, when button 60 is in its proximal position, cutting blade 40 is retracted within an interior opening of cutting tip 30 and handle 20, such that cutting edge 43 is absent from longitudinal cavity 36. As shown in FIGS. 7B and 7D, when pushbutton 60 is slid along an interior track of handle 20 from its proximal to its distal position, blade extension region 44, and in turn, the remainder of blade 40, is likewise slid forwards from its retracted to its deployed position. In particular, the top edge of cutting blade 40 is advanced along longitudinal channel 37 in the lower surface of cutting tip 30. In its deployed position, as best seen in FIG. 7D, cutting edge 43 is exposed within longitudinal cavity 36 of cutting tip 30.

A sixth preferred embodiment of the present invention is shown in FIGS. 8A-8E. In this embodiment, cutting tip 30 Includes an elongated lower portion 38, spring biased member 39, and independent side walls formed by wires 90. While in FIGS. 8A-8E the side walls are connected by a loop at the tip 91, they alternatively may be formed as two independent side wall structures. As best seen in FIGS. 8D and 8E, elongated lower portion 38 has the cross section of a spherical cap (i.e., a region of a sphere lying above a given plane). Cutting blade 40 and, in turn, cutting edge 43, extends downwardly from a planar bottom surface of elongated lower portion 38. In one embodiment, the side walls 90 are made from a bar, or wire, forming a loop 91 around a distal end of cutting tip 30, and attaching to opposing sides of spring biased member 39. As shown in FIGS. 8D and 8E, the combination of the circular cross-sections of the collinear portions of wire 90 with the spherical cap cross section of elongated lower portion 38 of cutting tip 30 collectively form an upper surface of cutting tip 30 that is substantially the outer surface of a torispherical dome (i.e., the surface obtained from the intersection of a spherical cap with a tangent torus, with the radius of the spherical cap forming the crown radius, and the radius of the torus forming the knuckle radius)—albeit with a small gap between the upper toroidal and spherical surfaces.

As with the two opposing wing-like members of the embodiments of FIGS. 3-7, the two opposing ends of the wire loop, proximate lower portion 38 of cutting tip 30, serve to laterally displace the digital nerves proximate the tendon sheath to be released, as the cutting tip is advanced lengthwise along the tendon sheath, inhibiting the digital nerves from becoming trapped inside the longitudinal channel beneath the lower surface of the cutting tip and, in turn, inhibiting the digital nerves from being inadvertently severed by the cutting blade. Moreover, as the two opposing wires and, in turn, the remainder of cutting tip 30 are advanced along opposing sides of the convex, arcuate outer surface of the tendon sheath to be released, they serve to self-center the cutting tip and, in turn, the cutting blade and cutting edge about the longitudinal center of the tendon sheath, and farther serve to inhibit the cutting tip from sliding laterally to either side as the cutting tip is advanced.

In operation, the spring biased member 39 in its resting state causes cutting blade 40, and in particular cutting edge 43, to remain within a recessed channel of cutting tip 30, formed by wires 90 (side edges of the channel) and planar bottom surface of elongated lower portion 38 (top edge of the channel). With downward pressure on the handle, the spring biased member 39 allows wire 90 to rise vertically, exposing cutting edge 43, as best seen in FIG. 8E and, in turn, moving cutting edge 43 from a guarded to a deployed position, relative to wires 90 and the previously-formed recessed channel.

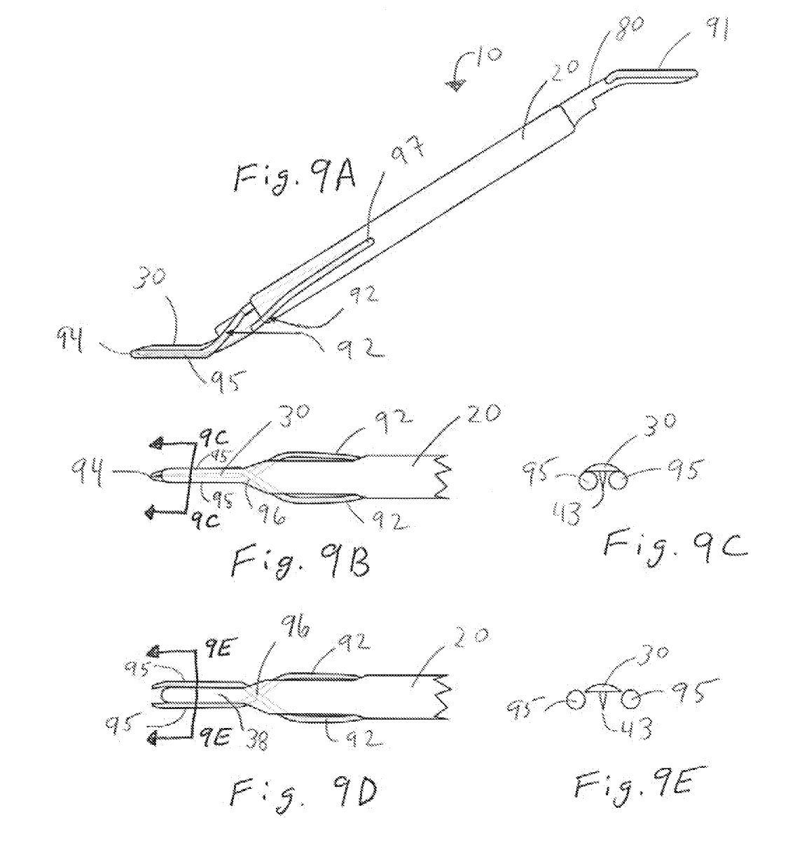

A seventh preferred embodiment of surgical tool 10 is shown in FIGS. 9A-9E. In this embodiment, two separate wires, rather than a wire loop as in the embodiment of FIGS. 8A-8E, is employed in the cutting tip, and operate in a manner similar to a pair of tongs. Two opposing wires each include a depressible region 92 proximate opposing sides of handle 20, a crossover region 96, and shield region 95. Shield region 95 of each opposing region join into a tapered tip 94. Each wire is operably attached to handle 20 at handle region 97. A single piece of wire, passing laterally through handle 20 at handle region 97, may alternatively be used.

As best seen in FIGS. 9C and 9E, cutting tip 30 again includes an elongated portion 38 which is substantially a hemispherical cap in cross section. Cutting blade 40 and, in turn, cutting edge 43, extends downwardly from a planar bottom surface of elongated lower portion 38 the combination of the circular cross-sections of the collinear shield regions 95 of the wires, together with the spherical cap cross section of elongated lower portion 38 of cutting tip 30, collectively form an upper surface of cutting tip 30 that is substantially the outer surface of a torispherical—albeit with a small gap between the upper toroidal and spherical surfaces.

When no external pressure is applied to depressible regions 92 of the wires, the wires meet at tapered tip 94. Moreover, the shield region 95 of each wire is proximate cutting blade 40 and cutting edge 43, providing a narrow, protective longitudinal cavity partially encasing cutting edge 43, as best seen in FIG. 9C. At this time, cutting blade 40 is accordingly in a guarded position. When inward pressure is simultaneously applied to both depressible regions 92 in a squeezing motion, the shield regions 95 of each wire separate from each other and, in turn, from cutting blade 40 and cutting edge 43. This, in turn, widens the effectively eliminates the narrow, protective longitudinal cavity that partially encased cutting edge 43, as best seen in FIG. 9E. At this time, cutting blade 40 is in its deployed position, and is free to extend downwardly, relative to shield region 95 of the opposing wires of cutting tip 30. This embodiment has the advantage of providing an instrument of smaller width in the resting, guarded state that can be introduced through a smaller skin incision.

An eight preferred embodiment of the present invention is shown in FIGS. 10A-10D. In this embodiment, the handle region comprises two opposing arms 21, moveable about pivot 23. The cutting tip comprises an elongated center portion 38, and two horizontally moveable jaws, or side walls 22. Cutting blade 40 and, in turn, cutting edge 43, extends downwardly from a bottom surface of elongated, center portion 38. As shown in FIGS. 10A and 10B, when opposing arms are spaced distally from each other, the jaws 22 are closed, the combination of the outer surfaces of jaws 22 and elongated center portion 38 collectively form an arcuate upper surface of the cutting tip. In this configuration, the outer surfaces of the jaws serve to laterally displace the digital nerves proximate the tendon sheath to be released, as the cutting tip is advanced lengthwise along the tendon sheath, inhibiting the digital nerves from becoming trapped inside the longitudinal channel beneath the lower surface of the cutting tip and, in turn, inhibiting the digital nerves from being inadvertently severed by the cutting blade. Moreover, as the two opposing jaws and, in turn, the remainder of cutting tip 30 are advanced along opposing sides of the convex, arcuate outer surface of the tendon sheath to he released, they serve to self-center the cutting tip and, in turn, the cutting blade and cutting edge about the longitudinal center of the tendon sheath, and further serve to inhibit the cutting tip from sliding laterally to either side as the cutting tip is advanced.

In operation, this embodiment of the present invention operates in a manner that is somewhat similar to a pair of pliers. A spring, or spring biased member (not shown in the drawings), serves to maintain handle arms 21 distally from each other and, in turn, serves to maintain jaws 22 in a closed configuration. When the handle arms are squeezed together, overcoming the opposing force of the spring or spring biased member, jaws 22 separate from each other, as shown in FIGS. 10C and 10D. This, in turn, exposes cutting edge 43, as best seen in FIG. 10D and accordingly moves cutting edge 43 from a guarded to a deployed position.

It is to be understood that even though numerous characteristics and advantages of the present inventive surgical tool have been set forth herein, together with the details of the structure and function of several preferred embodiments of the invention, the disclosure is illustrative only, and changes may be made in detail, especially in matters of shape, size and arrangement of parts within the principles of the invention to the full extent indicated by the broad general meaning of the terms in which the appended claims are expressed.

I claim:

1. A surgical tool for making subcutaneous incisions, the surgical tool comprising:
a handle region;
a cutting tip operably attached to the handle region, the cutting tip having a proximal end proximate the handle region, a distal end, a longitudinal axis extending from the proximal end to the distal end, and a longitudinal cavity between the proximal and distal ends; and
a cutting blade extending along at least a portion of the longitudinal axis of the cutting tip and extending downwardly from a bottom surface of the cutting tip into at least a portion of the longitudinal cavity, the cutting blade having a cutting edge that is substantially parallel to the longitudinal axis of the cutting tip.

2. The surgical tool according to claim 1, wherein the cutting tip has a substantially convex upper surface.

3. The surgical tool according to claim 1, wherein at least a portion of the distal end of the cutting tip comprises a substantially flat region for developing a horizontal
plane of dissection.

4. The surgical tool according to claim 1, wherein the cutting tip includes a bottom edge, and the cutting blade does not extend vertically below a plane perpendicular to the cutting blade and bisecting the bottom edge of the cutting tip.

5. The surgical tool according to claim 1, wherein an exposed height of the cutting blade, relative to the bottom surface of the cutting tip, is sized to substantially coincide with a desired incision depth in a tendon sheath.

6. The surgical tool according to claim 1, wherein at least a portion of the longitudinal cavity is substantially bilaterally symmetrical in cross section about the longitudinal axis of the cutting tip.

7. The surgical tool according to claim 1, wherein at least a portion of the longitudinal cavity has a surface contour substantially approximating a surface contour of a tendon sheath.

8. The surgical tool according to claim 1, wherein at least a portion of the longitudinal cavity has a surface contour that is substantially elliptical in cross section.

9. The surgical tool according to claim 1, wherein the cutting blade is moveable back and forth between a deployed position and a retracted position, and wherein at least a portion of the cutting blade is removed from the longitudinal cavity of the cutting tip when the cutting tip is in the retracted position.

10. The surgical tool according to claim 9, wherein movement of the cutting blade between the retracted and deployed positions is in a direction substantially perpendicular to the longitudinal axis of the cutting tip.

11. The surgical tool according to claim 9, wherein movement of the cutting blade between the retracted and deployed positions is in a direction substantially along the longitudinal axis of the cutting tip.

12. The surgical tool according to claim 9, wherein at least a portion of the cutting blade is carried within at least one of the cutting tip and the handle region when the cutting tip is in the retracted position.

13. The surgical tool according to claim 9, wherein the surgical tool further comprises a pushbutton operably attached to the cutting blade, and wherein depression of the pushbutton causes the cutting blade to move from the retracted position to the deployed position.

14. The surgical tool according to claim 13, wherein at least a portion of the pushbutton is integrally formed, with the cutting blade.

15. The surgical tool according to claim 9, wherein the surgical tool, further comprises a button operably attached to the cutting blade and slidably carried within at least one of the cutting tip and the handle region, and wherein sliding movement of the button from a first position to a second position causes the cutting blade to move from the retracted position to the deployed position,

16. The surgical tool according to claim 1, wherein the surgical tool further comprises a blade guard carried within at least one of the cutting tip and the handle region and moveable from a guarded position to an unguarded position, the blade guard covering at least a portion of the cutting edge of the cutting blade when in the guarded position, and exposing at least a portion of the cutting edge of the cutting blade when in the unguarded position.

17. The surgical tool according to claim 16, wherein the surgical tool further comprises a button operably attachable to the blade guard and slidably carried within at least one of the cutting tip and the handle region, and wherein sliding movement of the button from a first position to a second position causes the blade guard to move from the guarded position to the unguarded position.

18. The surgical tool according to claim 1, wherein the surgical tool further includes a dissection tip operably coupled to the handle region.

19. A surgical tool for making subcutaneous incisions through at least a portion of a tendon sheath having a substantially arcuate outer surface, the surgical tool comprising:
a handle region;
a cutting tip operably attached to the handle region, the cutting tip having a proximal end proximate the handle region, a distal end, a longitudinal axis extending from the proximal end to the distal end, an upper and a lower surface, a longitudinal cavity defined by at least a portion of the lower surface; and
a cutting blade having a deployed position wherein the cutting blade extends along at least a portion of the longitudinal axis of the cutting tip and extends downwardly from the bottom surface of the cutting tip into at least a portion of the longitudinal cavity;
wherein the lower surface of the cutting tip is configured to self-center the cutting tip as the cutting tip contacts the arcuate outer surface of the tendon sheath.

20. A surgical tool for making subcutaneous incisions through at least a portion of a tendon sheath proximate at least one of neural tissue and vascular tissue:
a handle region;
a cutting tip operably attached to the handle region, the cutting tip having a proximal end proximate the handle region, a distal end, a longitudinal axis extending from the proximal end to the distal end, an upper and a lower surface, and a longitudinal cavity defined by at least a portion of the lower surface; and
a cutting blade having a deployed position wherein the cutting blade extends along at least a portion of the longitudinal axis of the cutting tip and extends downwardly from the bottom surface of the cutting tip into at least a portion of the longitudinal cavity;
wherein the upper surface of the cutting tip is configured to displace at least one of the neural tissue and vascular tissue from a region proximate the cutting blade as the cutting tip is advanced along the arcuate outer surface of the tendon sheath.
